(19) United States
(12) Patent Application Publication
(10) Pub. No.: US 2003/0216426 A1
(43) Pub. Date: Nov. 20, 2003

(54) Treatment of cancer with mefloquine, its purified enantiomers, and mefloquine analogs

(75) Inventors: Dennis A. Carson, Del Mar, CA (US); Lorenzo M. Leoni, San Diego, CA (US); Howard B. Cottam, Escondido, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: REGENTS OF THE UNIVERSITY OF CALIFORNIA, Oakland, CA

(21) Appl. No.: 10/150,619

(22) Filed: May 17, 2002

(51) Int. Cl. A61K 31/4745; A61K 31/4709

(52) U.S. Cl. 514/305; 514/314

(57) ABSTRACT

Cancers, particularly solid tumors (e.g., breast, colon and ovarian cancers) and cancers of the hematologic system, e.g., hemopoietic cancers such as leukemias, lymphomas or myelomas, are treated by administration of a therapeutically effective amount of a compound having the formula (I):

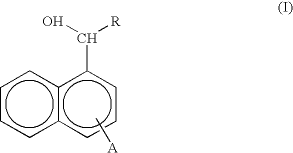

in which the quinoline ring is substituted by from one to three groups selected from halogen and trifluoromethyl (designated in the formula by “A”), and is optionally further substituted by one or more other moieties and R is (a) NR₁R₂ in which R₁ and R₂ are independently hydrogen or C₁-C₄ alkyl; (b) 2-piperidyl, (c) 2-pyridyl, and (d) 5-(ethyl or vinyl)-quinuclidin-4-yl; an enantiomer of such a compound; a pharmaceutically acceptable salts of such a compound or of an enantiomer thereof; a prodrug of such a compound or of an enantiomer thereof; a metabolite of such a compound or of an enantiomer thereof; and mixtures of two or more of the foregoing. A particularly preferred compound is mefloquine.

### FIELD AND BACKGROUND OF THE INVENTION

This invention relates to the treatment of cancer. More particularly, it relates to the treatment of cancers such as solid tumors and hematological malignancies. The former includes cancers such as breast, colon, and ovarian cancers. The latter include hematopoietic malignancies including leukemias, lymphomas and myelomas. This invention provides new, effective methods, compositions and kits for treatment and/or prevention of various types of cancer.

Hematological malignancies, such as leukemias and lymphomas, are conditions characterized by abnormal growth and maturation of hematopoietic cells.

Leukemias are generally neoplastic disorders of hematopoietic stem cells, and include adult and pediatric acute myeloid leukemias (AML), chronic myeloid leukemia (CML), acute lymphocytic leukemia (ALL), chronic lymphocytic leukemia (CLL), hairy cell leukemia and secondary leukemia. Myeloid leukemias are characterized by infiltration of the blood, bone marrow, and other tissues by neoplastic cells of the hematopoietic system. CLL is characterized by the accumulation of mature-appearing lymphocytes in the peripheral blood and is associated with infiltration of bone marrow, the spleen and lymph nodes.

Specific leukemias include acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia.

Lymphomas are generally neoplastic transformations of cells that reside primarily in lymphoid tissue. Among lymphomas, there are two major distinct groups: non-Hodgkin's lymphoma (NHL) and Hodgkin's disease. Lymphomas are tumors of the immune system and generally are present as both T cell- and as B cell-associated disease. Bone marrow, lymph nodes, spleen and circulating cells are all typically involved. Treatment protocols include removal of bone marrow from the patient and purging it of tumor cells, often using antibodies directed against antigens present on the tumor cell type, followed by storage. The patient is then given a toxic dose of radiation or chemotherapy and the purged bone marrow is then reinfused in order to repopulate the patient's hematopoietic system.

Other hematological malignancies include myelodysplastic syndromes (MDS), myeloproliferative syndromes (MPS) and myelomas, such as solitary myeloma and multiple myeloma. Multiple myeloma (also called plasma cell myeloma) involves the skeletal system and is characterized by multiple tumorous masses of neoplastic plasma cells scattered throughout that system. It may also spread to lymph nodes and other sites such as the skin. Solitary myeloma involves solitary lesions that tend to occur in the same locations as multiple myeloma.

Hematological malignancies are generally serious disorders, resulting in a variety of symptoms, including bone marrow failure and organ failure. Treatment for many hematological malignancies, including leukemias and lymphomas, remains difficult, and existing therapies are not universally effective. While treatments involving specific immunotherapy appear to have considerable potential, such treatments have been limited by the small number of known malignancy-associated antigens. Moreover the ability to detect such hematological malignancies in their early stages can be quite difficult depending upon the particular malady. Accordingly, there remains a need in the art for improved methods for treatment of hematological malignancies such as B cell leukemias and lymphomas and multiple myelomas. The present invention fulfills these and other needs in the field.

Other cancers are also of concern, and represent similar difficulties insofar as effective treatment is concerned. Such cancers include those characterized by solid tumors. These include, for instance, epidermoid and myeloid tumors, lung cancers, ovarian cancers, breast cancers and colon cancers. Still other types of cancers of concern, and to which this invention relates, include endometrial tumors, bladder cancer, pancreatic cancer, testicular cancer, renal cancers, cancer of the esophagus, and tumors of the central nervous system such as brain tumors. The present invention is generally directed to compositions and methods for the treatment of such cancers, and others.

### BRIEF SUMMARY OF THE INVENTION

In brief, this invention provides a method of treating cancer, comprising administering to a patient in need thereof a therapeutically effective amount of a composition comprising a compound selected from mefloquine and mefloquine analogs (as defined herein), enantiomers of mefloquine or of its analogs, pharmaceutically acceptable salts of mefloquine, of its analogs or of enantiomers of either; prodrugs of mefloquine, of its analogs, or of enantiomers of either; metabolites of mefloquine, of its analogs, or of enantiomers of either; and mixtures thereof.

The invention further provides compositions or formulations for treating such cancers that contain an effective amount for this purpose, of a compound selected from those mentioned above, or a mixture thereof, as well as compositions that further contain other agents for treating such cancers.

In addition, the invention provides kits for treating such cancers that include a composition containing an effective amount, for this purpose, of a compound selected from mefloquine, enantiomers of mefloquine, pharmaceutically acceptable salts of mefloquine or of its enantiomers; prodrugs of mefloquine or of its enantiomers; metabolites of mefloquine or of its enantiomers; or a mixture of such compounds.

These and other aspects of the invention will be discussed in more detail below.

### BRIEF DESCRIPTION OF THE DRAWINGS

FIG. 1 is a graphical depiction of selective cytotoxicity of racemic mefloquine to chronic lymphocytic leukemia (CLL) cells and to normal control lymphocytes.

FIG. 2 depicts a flow cytometric analysis of the effect of racemic mefloquine and its isolated stereoisomers on CLL cells.

FIG. 3 is a graphical depiction of the effect of the isolated stereoisomer (−)-mefloquine on cell viability.

FIG. 4 is a graphical depiction of the effect of mefloquine on some solid tumors.

### DETAILED DESCRIPTION OF THE INVENTION

This invention relates to a method of treating cancer, comprising administering to a patient in need thereof a therapeutically effective amount, for this purpose, of a composition comprising a compound having the formula (I):

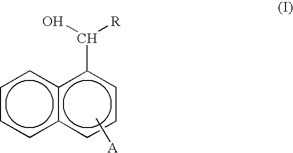

in which the quinoline ring is substituted by from one to three groups selected from halogen and trifluoromethyl (designated in the formula by “A”), and is optionally further substituted by one or more other moieties, and R is (a) NR₁R₂ in which R₁ and R₂ are independently hydrogen or C₁-C₄ alkyl; (b) 2-piperidyl, (c) 2-pyridyl, and (d) 5-(ethyl or vinyl)-quinuclidin-4-yl; an enantiomer of such a compound; a pharmaceutically acceptable salt of such a compound or of an enantiomer thereof; a prodrug of such a compound or of an enantiomer thereof; a metabolite of such a compound or of an enantiomer thereof; and mixtures of two or more of the foregoing.

In other aspects, the invention relates to such compositions and to kits containing such compositions for use in treating cancer in patients.

The compounds above have from one to three groups selected from halogen and trifluoromethyl substituted on the quinoline ring (designated as “A”). Typically such groups are substituted at the 2-, 6-, and/or 8-positions. By “halogen” is meant a chlorine, fluorine, bromine or iodine atom. Of the halogen substituents, chlorine is preferred. Preferred groups for R are 2-piperidyl, 2-pyridyl, 5-(ethyl or vinyl)-quinuclid-2-yl, and NR₁R₂ wherein one or both of R₁ and R₂ are C₃-C₄ alkyl. In addition to the halogen and/or trifluoromethyl groups, the quinoline ring may also be substituted, as is known in the art. with one or more other groups such as lower alkyl (for instance, methyl), lower alkoxy (for instance, methoxy), phenyl, halophenyl, trifluoromethylphenyl, other substituted phenyl groups, and the like.

One currently known and commercially available compound of this class is mefloquine.

Mefloquine is a 4-quinolinemethanol derivative with the specific chemical name of (R*, S*)-(±)-alpha-2-piperidinyl-2,8-bis (trifluoromethyl)-4-quinolinemethanol. It is a 2-aryl substituted chemical structural analog of quinine. Typically it is available and is used in the form of its hydrochloride salt. Mefloquine hydrochloride is a white to almost white crystalline compound, soluble in ethanol and slightly soluble in water.

Mefloquine has the structural formula (II):

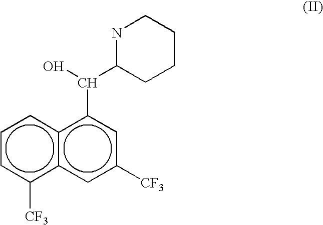

The current use of mefloquine is as an antiparasitic treatment for malaria. It is available from Roche under the trademark Lariam®. Since mefloquine has two stereocenters, there are four possible enantiomers: RS(+), SR(−), RR, and SS.

Mefloquine is not generally known as a treatment for any form of cancer, although researchers at the Hebrew University of Jerusalem have reported that mefloquine, in combination with certain other drugs, demonstrated the property of modulating the resistance pump P-glycoprotein in leukemia cells [Ayesh et al., Biochimica et Biophysica Acta 1316, 8 (1996); Lan et al., Cancer Chemother. Pharmacol. 38, 181 (1996); Shao et al., Biochimica et Biophysica Acta 1360, 30 (1997)]. The recently published United States patent application 2002-00022032 of Patrick Curry et al. includes mefloquine in an extremely long list of photosensitizers that are said to be useful in a combined immuno-adjuvant photodynamic therapy for treatment and prevention of metastatic cancer (though no data is presented for any such combination that includes mefloquine).

Other compounds in the class of mefloquine analogs are described in literature and patents. For example, Schmidt et al., Antimicrobial Agents and Chemotherapy 13: 1011 (1978) describes a number of such compounds (including enantiomers of mefloquine) that were screened for anti-malarial activity. Some others are disclosed, for instance in Buchman et al., J.A.C.S. 68: 2710 (1946), Rothe et al., J. Med. Chem. 11: 366 (1968), Ison et al., J. Invest. Dermatol. 52: 193 (1969), and Ohnmacht et al., J. Med. Chem. 14: 926 (1971). Schmidt et al., supra and Grethe et al., U.S. Pat. No. 3,953,453, disclose some quinuclidinyl compounds of formula (I). All these references are hereby incorporated by reference herein.

Another anti-malarial member of the family of quinine-type compounds, hydroxychloroquine, is useful as a therapeutic agent in systemic lupus and rheumatoid arthritis and has been shown to induce apoptosis to apoptosis in peripheral blood T lymphocytes [Meng et al., Arthritis Rheum. 40: 927 (1997)], but the mechanism of this induction remains unclear. Hydroxychloroquine has been recently shown to be active against CLL cells, [Lagneaux et al., Br. J. Haematol. 112: 344 (2001)], but at concentrations (30 μg/ml and above) that are not feasible for use in vivo due to toxicity.

According to the present invention, compounds of formula (I) are used for treating cancers in general, particularly a solid tumor or a hematological or hemopoietic cancer, by administration to a patient or subject in need of treatment, in an effective amount. The compound may be administered as such, or in the form of an equivalent material such as a prodrug, a metabolite, an enantiomer, or a salt of any of these, or a mixture of two or more such forms.

### Definitions

As used herein, the terms below have these definitions.

“Cancer”, and specific types of cancers such as “leukemia”, “lymphoma” and “myeloma” are defined as described in the introductory portion of this patent application and as generally understood by those skilled in the art.

A “pharmaceutically acceptable” component is one that is suitable for use with humans and/or animals without undue adverse side effects (such as toxicity, irritation, and allergic response) commensurate with a reasonable benefit/risk ratio.

A “safe and effective amount” refers to the quantity of a component that is sufficient to yield a desired therapeutic response without undue adverse side effects (such as toxicity, irritation, or allergic response) commensurate with a reasonable benefit/risk ratio when used in the manner of this invention. By “therapeutically effective amount” is meant an amount of a component effective to yield the desired therapeutic response, for example, an amount effective to delay the growth of a cancer or to cause a cancer to shrink or not metastasize. The specific safe and effective amount or therapeutically effective amount will vary with such factors as the particular condition being treated, the physical condition of the patient, the type of mammal being treated, the duration of the treatment, the nature of concurrent therapy (if any), and the specific formulations employed and the structure of the compounds or its derivatives.

A “pharmaceutically acceptable salt” is a salt of the compound in question that is pharmaceutically acceptable as that term is defined above. Examples of pharmaceutically acceptable base addition salts include sodium, potassium, calcium, ammonium, organic amino, magnesium salts, and the like. When compounds of the present invention contain relatively basic functionalities, salts can be obtained by addition of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galacturonic acids and the like (see, for example, Berge et al., “Pharmaceutical Salts”, Journal of Pharmaceutical Science, 1977, 66, 1-19).

A “pharmaceutically acceptable carrier” is a carrier, such as a solvent, suspending agent or vehicle, for delivering the compound or compounds in question to the animal or human. The carrier may be liquid or solid and is selected with the planned manner of administration in mind. Liposomes are also a pharmaceutical carrier. As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Carriers for use in the compositions of this invention are described in more detail below

A “prodrug” is a compound that readily undergoes chemical changes under physiological conditions (i.e., in the patient's body) to provide the compounds used in the present invention (e.g., to produce mefloquine, a mefloquine enantiomer, a mefloquine salt or a mefloquine metabolite) in situ. Additionally, prodrugs can be converted to the compounds of the present invention by chemical or biochemical methods ex vivo. For example, prodrugs can be slowly converted to the compounds of the present invention when placed in a transdermal patch reservoir with a suitable enzyme or chemical reagent.

Typical prodrugs are compounds that readily become metabolized or converted to the active compound of formula (I) through, for instance cleavage of an ester, amine or acyl group.

A “metabolite” is a compound formed in the patient's system from a compound of this invention. Two metabolites of mefloquine have been identified in humans. The main metabolite is 2,8-bis-trifluoromethyl-4-quinoline carboxylic acid. The other metabolite is an alcohol that typically is present in only minute amounts. Mefloquine has an excellent bioavailability (80%), and can accumulate at micromolar levels in the plasma. The major mefloquine metabolite (the carboxylic acid derivative) accumulates in the plasma at even higher levels (up to 10 μM, as shown in Table 1 below).

TABLE I
Mean (SD) trough concentrations of racemic
mefloquine (rac MQ) the separate enantiomers (RS and
SR) and the carboxylic acid metabolite in plasma, serum
and whole blood in ten volunteers at steady state

| Concentration | Plasma | Serum | Whole Blood |
|---|---|---|---|
| Rac-MQ (μmol · 1⁻¹) | 2.17 (0.83) | 1.98 (0.80) | 2.04 (0.71) |
| (+.) RS (μmol · 1⁻¹) | 0.26 (0.11) | 0.26 (0.89) | 0.35 (0.11) |
| (−).SR (μmol · 1⁻¹) | 1.91 (0.75) | 1.72 (0.74) | 1.69 (0.64) |
| Metabolite (μmol · 1⁻¹) | 9.80 (5.07) | 9.44 (5.00) | 4.89 (2.58) |

Results obtained with healthy volunteers dosed at 250 mg once weekly for 16 weeks; results show the steady state levels at week 16¹.

### Enantiomers

As can readily be seen from its structure, mefloquine contains two asymmetric carbon atoms and thus may have four enantiomers: RS, SR, RR and SS. The RS, SR pair is referred to in the original literature report of the chemistry as (−) erythro and (+) erythro (see F. I. Carroll and J. T. Blackwell, J. Med. Chem., 1974, 17, 210-219). The commercial material used clinically and sold under the trademark LARIAM® is a racemic mixture of the erythro pair, RS and SR, and contains none of the other two enantiomers, known as the threo pair (RR and SS). Separation and purification of the erythro pair of isomers is accomplished by fractional crystallization of an acid salt of mefloquine according to the procedure reported in the above reference. Mefloquine displays stereoselective pharmacodynamics and biochemistry. The (−) mefloquine does not seem to pass the blood brain barrier and accumulates at higher levels in the plasma. The (+) mefloquine has been shown to accumulate in the brain, but has a lower plasma level. The carboxylic acid metabolite will not penetrate the brain barrier.

### Compositions and Formulations

For use in this invention, the active compound of formula (I), for instance, racemic mefloquine, an enantiomer of mefloquine, a prodrug of either the racemic mixture or of a stereoisomer, a metabolite of either the racemic mixture or of a stereoisomer, or a salt of any of these, is included or formulated into a composition for packing, storage, shipment and administration. The compositions will contain one or more pharmaceutically acceptable carriers and may also contain other therapeutically active ingredients as well as adjuvants and other ingredients that may be found in pharmaceutical compositions.

Thus, compounds of this invention can be formulated with a pharmaceutically acceptable carrier for administration to a subject. While any suitable carrier known to those of ordinary skill in the art may be employed in the pharmaceutical compositions of this invention, the type of carrier will vary depending on the mode of administration. The pharmaceutical composition is typically formulated such that the compound in question is present in a therapeutically effective amount, i.e., the amount of compound required to achieve the desired effect in terms of treating a subject.

For preparing pharmaceutical compositions, the pharmaceutically acceptable carriers can be either solid or liquid. Solid form preparations include powders, tablets, pills, capsules, cachets, suppositories, and dispersible granules. A solid carrier can be one or more substance that may also act as diluents, flavoring agents, binders, preservatives, tablet disintegrating agents, or an encapsulating material. For example, one pill that contains mefloquine and had been sold for anti-malarial use contains, in addition to the (racemic) mefloquine, the inert ingredients ammonium-calcium alginate, cornstarch, crospovidone, lactose, magnesium stearate, microcrystalline cellulose, poloxamer #331, and talc.

In powders, the carrier is a finely divided solid that is in a mixture with the finely divided active component. In tablets, the active component is mixed with the carrier having the necessary binding properties in suitable proportions and compacted in the shape and size desired.

Suitable carriers for the solid compositions of this invention include, for instance, magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Alternatively the mefloquine may be prepared in a form with an encapsulating material as a carrier providing a capsule in which the active component, with or without other carriers, is surrounded by a carrier, which is thus in association with it. Similarly, cachets and lozenges are included. Tablets, powders, capsules, pills, cachets, and lozenges can be used as solid dosage forms suitable for oral administration.

Liquid form preparations include solutions, suspensions, and emulsions, for example, water or water/propylene glycol solutions or suspensions. Aqueous suspensions suitable for oral use can be made by dispersing the finely divided compound in water with viscous material, such as natural or synthetic gums, resins, methylcellulose, sodium carboxymethylcellulose, and other well-known suspending agents. For parenteral injection, liquid preparations can be formulated in solution in aqueous polyethylene glycol solution. In certain embodiments, the pharmaceutical compositions are formulated in a stable emulsion formulation (e.g., a water-in-oil emulsion or an oil-in-water emulsion) or an aqueous formulation that preferably comprises one or more surfactants. Suitable surfactants well known to those skilled in the art may be used in such emulsions. In one embodiment, the composition comprising the compound in question is in the form of a micellar dispersion comprising at least one suitable surfactant. The surfactants useful in such micellar dispersions include phospholipids. Examples of phospholipids include: diacyl phosphatidyl glycerols, such as: dimyristoyl phosphatidyl glycerol (DPMG), dipalmitoyl phosphatidyl glycerol (DPPG), and distearoyl phosphatidyl glycerol (DSPG); diacyl phosphatidyl cholines, such as: dimyristoyl phosphatidylcholine (DPMC), dipalmitoyl phosphatidylcholine (DPPC), and distearoyl phosphatidylcholine (DSPC); diacyl phosphatidic acids, such as: dimyristoyl phosphatidic acid (DPMA), dipalmitoyl phosphatidic acid (DPPA), and distearoyl phosphatidic acid (DSPA); and diacyl phosphatidyl ethanolamines such as: dimyristoyl phosphatidyl ethanolamine (DPME), dipalmitoyl phosphatidyl ethanolamine (DPPE), and distearoyl phosphatidyl ethanolamine (DSPE). Other examples include, but are not limited to, derivatives of ethanolamine (such as phosphatidyl ethanolamine, as mentioned above, or cephalin), serine (such as phosphatidyl serine) and 3′-O-lysyl glycerol (such as 3′-O-lysyl-phosphatidylglycerol).

Also included in compositions for use in this invention are solid form preparations that are intended to be converted, shortly before use, to liquid form preparations for oral administration. Such liquid forms include solutions, suspensions, and emulsions. These preparations may contain, in addition to the active compound, colorants, flavors, stabilizers, buffers, artificial and natural sweeteners, dispersants, thickeners, solubilizing agents, and the like.

The compositions of the invention may also be in the form of controlled release or sustained release compositions as known in the art, for instance, in matrices of biodegradable or non-biodegradable injectable polymeric microspheres or microcapsules, in liposomes, in emulsions, and the like.

Compositions of this invention for use in treating cancers may contain other therapeutically active ingredients such as other therapeutic agents for treating the cancers in question. Conversely, the therapeutic agents may be incorporated into other types of cancer treatment agents such as vaccines.

Compositions of these compounds may also contain one or more compounds that possess adjuvant activity. Such compounds include, for instance, aluminum hydroxide, mineral oils, alum-based adjuvants (e.g., Alhydrogel, Rehydragel, aluminum phosphate, Algammulin, aluminum hydroxide); oil based adjuvants (Freund's Incomplete Adjuvant and Complete Adjuvant (Difco Laboratories, Detroit, Mich.), Specol, RIBI, TiterMax, Montanide ISA50 or Seppic MONTANIDE ISA 720); nonionic block copolymer-based adjuvants, cytokines (e.g., GM-CSF or Flat3-ligand); Merck Adjuvant 65 (Merck and Company, Inc., Rahway, N.J.); AS-2 (SmithKline Beecham, Philadelphia, Pa.); salts of calcium, iron or zinc; an insoluble suspension of acylated tyrosine; acylated sugars; cationically or anionically derivatized polysaccharides; polyphosphazenes; biodegradable microspheres; monophosphoryl lipid A; aminoalkyl glucosaminide phosphates; and saponins such as Quil A and QS-21. Cytokines, such as GM-CSF or interleukin-2, -7, or -12, may also be used as adjuvants.

### Administration

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The compounds (in the form of their compositions) are administered to patients by the usual means known in the art, for example, orally or by injection, infusion, infiltration, irrigation, and the like. For administration by injection and/or infiltration or infusion, the compositions or formulations according to the invention may be suspended or dissolved as known in the art in a vehicle suitable for injection and/or infiltration or infusion. Such vehicles include isotonic saline, buffered or unbuffered and the like. Depending on the intended use, they also may contain other ingredients, including other active ingredients, such as isotonicity agents, sodium chloride, pH modifiers, colorants, preservatives, antibodies, enzymes, antibiotics, antifungals, antivirals, other anti-infective agents, and/or diagnostic aids such as radio-opaque dyes, radiolabeled agents, and the like, as known in the art. However, the compositions of this inventions may comprise no more than a simple solution or suspension of a compound or a pharmaceutically acceptable salt of a compound, in distilled water or saline.

Alternatively, the therapeutic compounds may be delivered by other means such as intranasally, by inhalation, or in the form of liposomes, nanocapsules, vesicles, and the like. Compositions for intranasal administration usually take the form of drops, sprays containing liquid forms (solutions, suspensions, emulsions, liposomes, etc.) of the active compounds. Administration by inhalation generally involves formation of vapors, mists, dry powders or aerosols, and again may include solutions, suspensions, emulsions and the like containing the active therapeutic agents

Routes and frequency of administration of the therapeutic compositions described herein, as well as dosage, will vary from individual to individual, and may be readily established using standard techniques. Preferably, between 1 and 10 doses may be administered over a 52-week period. A suitable dose is an amount of a compound that, when administered as described above, is capable of killing or slowing the growth of, cancers or cancer cells.

In general, an appropriate dosage and treatment regimen provides the active compound(s) in an amount sufficient to provide therapeutic and/or prophylactic benefit. Such a response can be monitored by establishing an improved clinical outcome (e.g., more frequent remissions, complete or partial, or longer disease-free survival) in treated patients as compared to non-treated patients.

A therapeutic amount of a compound of formula (I), as mentioned above, means an amount effective to yield the desired therapeutic response, for example, an amount effective to delay the growth of a cancer or to cause a cancer to shrink or not metastasize. If what is administered is not the compound itself but an enantiomer, prodrug, salt or metabolite of the compound, then the term “therapeutically effective amount” means an amount of such material that produces in the patient the same blood concentration of the compound in question that is produced by the administration of a therapeutically effective amount of the compound itself. For instance, as shown in the examples below, mefloquine now has been shown to be effective against CLL cells at concentrations of 10 μM and below. Accordingly, one therapeutically effective amount of mefloquine is that which produces a blood concentration of 10 μM mefloquine in a patient. Similarly, if an enantiomer, prodrug or metabolite of mefloquine, or a salt of mefloquine or of any of these other compounds, is being administered, then one therapeutically effective amount of such a compound is that amount that produces a blood concentration of mefloquine in a patient.

Patients that can be treated with the compounds of formula (I), and the pharmaceutically acceptable salts, prodrugs, enantiomers and metabolites of such compounds, according to the methods of this invention include, for example, patients that have been diagnosed as having lung cancer, bone cancer, pancreatic cancer, skin cancer, cancer of the head and neck, cutaneous or intraocular melanoma, uterine cancer, ovarian cancer, rectal cancer or cancer of the anal region, stomach cancer, colon cancer, breast cancer, gynecologic tumors (e.g., uterine sarcomas, carcinoma of the fallopian tubes, carcinoma of the endometrium, carcinoma of the cervix, carcinoma of the vagina or carcinoma of the vulva), Hodgkin's disease, cancer of the esophagus, cancer of the small intestine, cancer of the endocrine system (e.g., cancer of the thyroid, parathyroid or adrenal glands), sarcomas of soft tissues, cancer of the urethra, cancer of the penis, prostate cancer, chronic or acute leukemia, solid tumors of childhood, lymphocytic lymphomas, cancer of the bladder, cancer of the kidney or ureter (e.g., renal cell carcinoma, carcinoma of the renal pelvis), or neoplasms of the central nervous system (e.g., primary CNS lymphoma, spinal axis tumors, brain stem gliomas or pituitary adenomas).

In further aspects of the present invention, the compositions described herein may be used to treat hematological malignancies including adult and pediatric AML, CML, ALL, CLL, myelodysplastic syndromes (MDS), myeloproliferative syndromes (MPS), secondary leukemia, multiple myeloma, Hodgkin's lymphoma and Non-Hodgkin's lymphomas.

Within such methods, pharmaceutical compositions are typically administered to a patient. As used herein, a “patient” refers to any warm-blooded animal, preferably a human. A patient may or may not be afflicted with a hematological malignancy. Accordingly, the above pharmaceutical compositions may be used to prevent the development of a malignancy, or delay it s appearance or reappearance, or to treat a patient afflicted with a malignancy. A hematological malignancy may be diagnosed using criteria generally accepted in the art. Pharmaceutical compositions may be administered either prior to or following surgical removal of primary tumors and/or treatment such as administration of radiotherapy or conventional chemotherapeutic drugs, or bone marrow transplantation (autologous, allogeneic or syngeneic).

The compositions provided herein may be used alone or in combination with conventional therapeutic regimens such as surgery, irradiation, chemotherapy and/or bone marrow transplantation (autologous, syngeneic, allogeneic or unrelated).

Kits for administering the compounds may be prepared containing a composition or formulation of the compound in question, or an enantiomer, prodrug, metabolite, or pharmaceutically acceptable salt of any of these, together with the customary items for administering the therapeutic ingredient.

### EXAMPLES

The invention is further illustrated by the examples that follow. However, it should be noted that these are presented as examples of the invention, and do not limit it in any way.

### Example 1

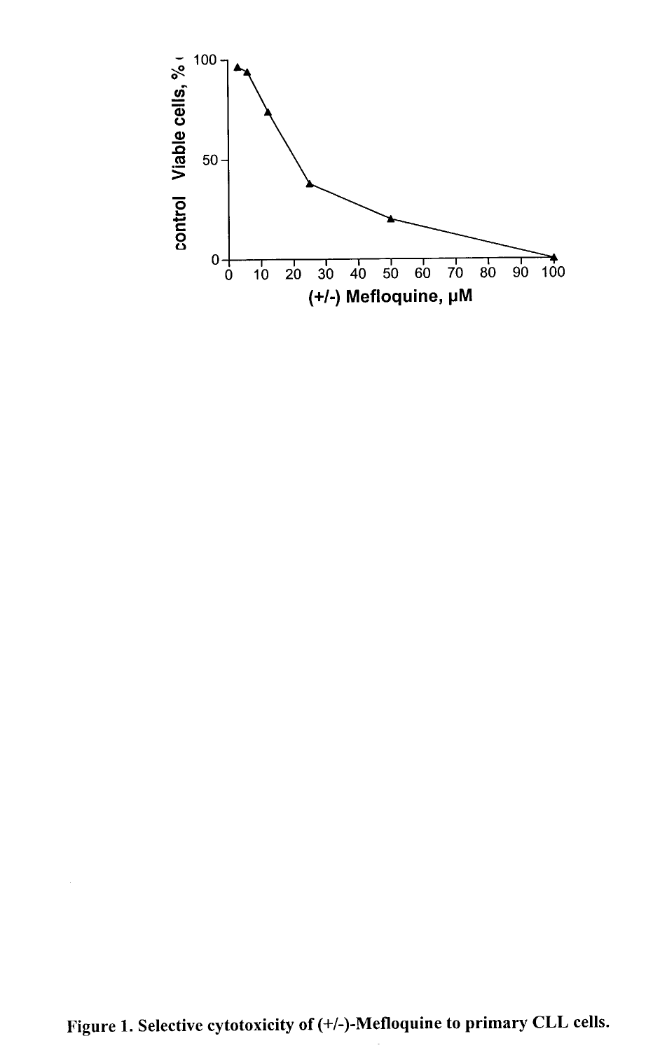

Chronic lymphocytic leukemia cells (CLL) isolated from blood of two different CLL patients (CLL #1, CLL #2), and control normal lymphocytes (PBL) isolated from a healthy volunteer were exposed to concentrations of 5, 10, 25, 50 and 100 μM, of the racemic mix of mefloquine (+/−). After 48 hours exposure, the number of viable cells was determined by dye exclusion and flow cytometric analysis. The results are shown in FIG. 1, where the x axis represents the mefloquine concentration and they axis represents the number of viable cells, normalized to the untreated controls. The (+/−)-mefloquine was found to be inducing potent apoptosis in CLL cells at 10 μM, but not against normal lymphocytes.

The (+/−)-mefloquine was also found to be able to induce apoptosis against myeloma cell lines RPM18226 (IC₅₀ of 10-20 μM).

### Example 2

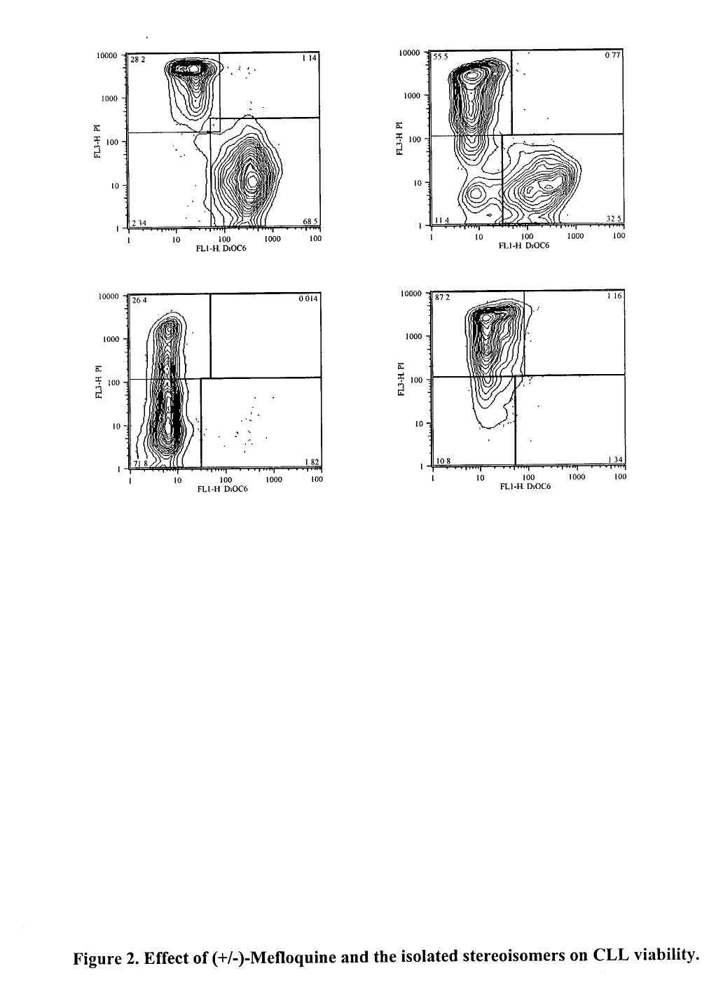

Chronic lymphocytic leukemia cells (CLL) isolated from blood of CLL patients were exposed to 10 μM of the racemic mix of mefloquine (+/−), the isolated mefloquine enantiomers (+) and (−), or vehicle alone (“untreated”). After 24 hours exposure, the percentage of living (lower-right corner), apoptotic (lower-left corner) and dead (higher-left corner) were analyzed by flow cytometry using propidium iodide (y axis) and DiOC6 (x axis). The results, showing the activity of the (+) and (−) enantiomers against CLL cells, are seen in FIG. 2.

### Example 3

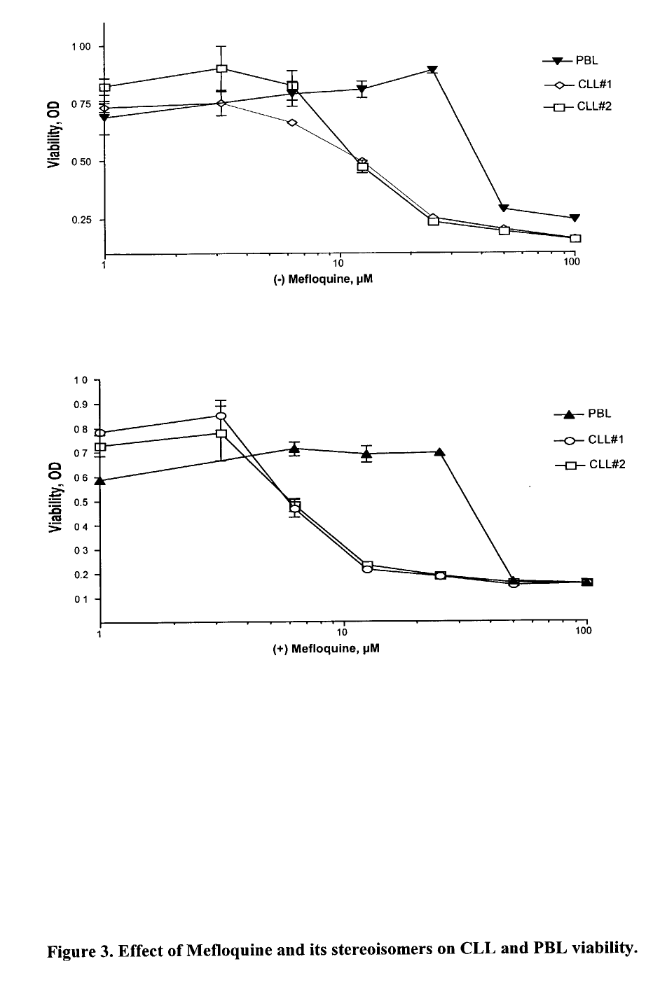

Lymphocytes isolated from blood of CLL patients (CLL) and from normal healthy volunteer (PBL) were exposed to various concentrations of purified (−)-mefloquine ranging from 1.0 to 10.0 μM. After 24 hours exposure, the number of viable cells was determined by dye exclusion and flow cytometric analysis. The results are shown in FIG. 3. They axis represents the number of viable cells, normalized to the untreated controls.

### Example 4

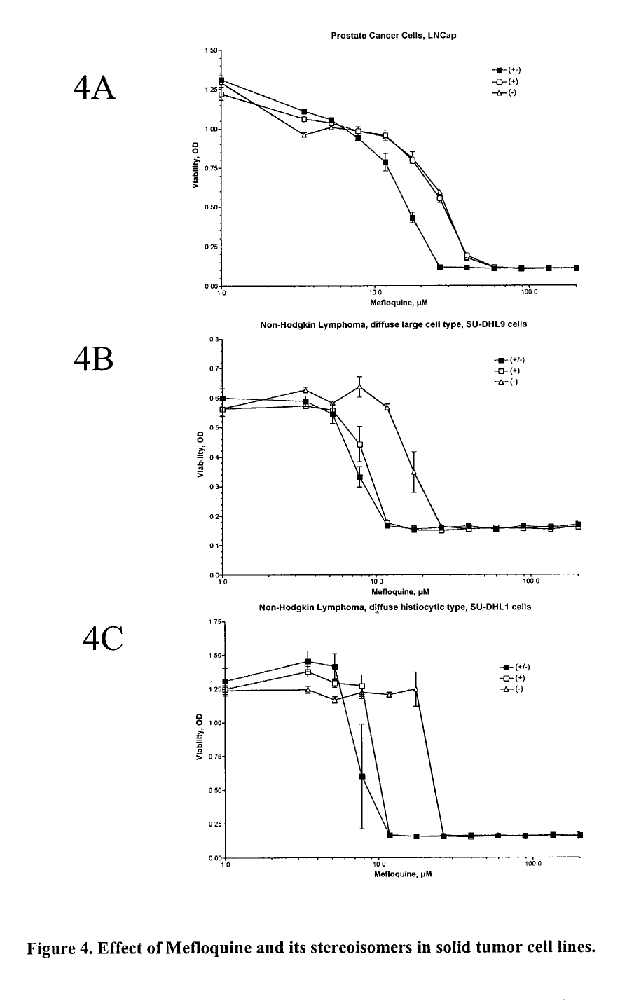

Prostate cancer cells (LNCap, 4A), and two different lymphoma cell lines (SU-DHL9, 4B, and SU-DHL1, 4C) were exposed to various concentrations from 200 to 1 μM of racemic mefloquine (+/−, closed symbol), and the purified stereoisomers (−)-mefloquine (open triangles) and (+)-mefloquine (open squares). After 72 hours exposure, the viability of the cells (y axis) was determined by the rate of conversion of the formazan salt measuring the optical density (OD) at 570 nm (MTT assay). The results are shown in FIG. 4.

### Example 5

The indicated cell lines were exposed to various concentrations from 200 to 1 μM of racemic mefloquine (+/−), and the purified stercoisomer (−)-mefloquine. After 72 hours exposure, the viability of the cells was determined by the rate of conversion of the formazan salt measuring the optical density (OD) at 570 nm (MTT assay). The IC₅₀ is the concentration of mefloquine that reduce cell viability to 50% from the untreated control.

The results are shown in the following Table 2.

TABLE 2
Effect of racemic mefloquine (+/−) and its stereoisomer (−)-
mefloquine in tumor cell lines.

| | | (+/−) Mefloquine, | (−) Mefloquine, |
|---|---|---|---|
| Cell line | Tumor type | IC₅₀ (μM) | IC₅₀ (μM) |
| MCF7 | Breast | 10 | 20 |
| MDA-231 | Breast | 20 | 40 |
| HCT-116 | Colon | 6 | 12 |
| LNCap | Prostate | 12 | 25 |
| SU-DHL1 | Lymphoma | 8 | 20 |
| SU-DHL9 | Lymphoma | 7 | 15 |
| 10C9 | Lymphoma | 9 | 20 |
| RAJI | Lymphoma | 12 | 30 |
| RPMI8226 | Myeloma | 8 | 15 |
| U266 | Myeloma | 11 | 25 |

It is understood that the examples and embodiments described herein are for illustrative purposes only and that various modifications or changes in light thereof will be suggested to persons skilled in the art and are to be included within the spirit and purview of this application and scope of the appended claims.

All publications, patents, and patent applications cited herein are hereby incorporated by reference in their entirety for all purposes.

What is claimed is:

1. A method of treating cancer comprising administering to a patient in need thereof a therapeutically effective amount of a composition comprising a compound having the formula (I): 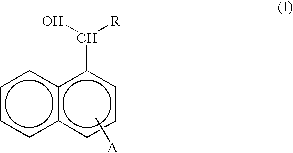
in which A represents from one to three groups selected from halogen and trifluoromethyl, and in which the quinoline ring is optionally further substituted by one or more other moieties, and R is (a) NR₁R₂ in which R₁ and R₂ are independently hydrogen or C₁-C₄ alkyl; (b) 2-piperidyl, (c) 2-pyridyl, and (d) 5-(ethyl or vinyl)-quinuclidin-4-yl; an enantiomer of such a compound; a pharmaceutically acceptable salt of such a compound or of an enantiomer thereof; a prodrug of such a compound or of an enantiomer thereof; a metabolite of such a compound or of an enantiomer thereof; and mixtures of two or more of the foregoing.

2. A method according to claim 1 in which A represents from one to three chloro or trifluoromethyl groups, and R is (a) NR₁R₂ in which R₁ and R₂ are independently hydrogen or C₃-C₄ alkyl; (b) 2-piperidyl, (c) 2-pyridyl, and (d) 5-(ethyl or vinyl)-quinuclidin-4-yl.

3. A method according to claim 1 in which the quinoline ring is further substituted by a methoxy, methyl, phenyl, halophenyl or trifluoromethyl group.

4. A method according to claim 1 in which the quinoline ring is substituted by from one to three groups selected from halogen and trifluoromethyl and is not further substituted.

5. A method according to claim 1 in which the compound is selected from mefloquine, enantiomers of mefloquine; prodrugs of mefloquine or of its enantiomers; metabolites of mefloquine or of its enantiomers; pharmaceutically acceptable salts of mefloquine, of mefloquine enantiomers, of mefloquine prodrugs or of mefloquine metabolites, and mixtures thereof.

6. A method according to claim 1 in which the cancer is a cancer of the hematological system.

7. A method according to claim 1 in which the cancer is a cancer of the hematopoietic system.

8. A method according to claim 1 in which the cancer is selected from leukemias, myelomas and lymphomas.

9. A method according to claim 1 in which the cancer is a cancer that is in the form of a solid tumor.

10. A method according to claim 1 in which the cancer is selected from breast cancer, colon cancer and ovarian cancer

11. A method according to claim 1 comprising administering mefloquine to a patient.

12. A method according to claim 1 comprising administering an enantiomer of mefloquine to a patient.

13. A method according to claim 1 comprising administering to a patient a prodrug of mefloquine or of a mefloquine enantiomer.

14. A method according to claim 1 comprising administering to a patient a metabolite of mefloquine or of a mefloquine enantiomer.

15. A method according to claim 1 comprising administering to a patient a salt of (a) mefloquine, (b) a mefloquine enantiomer, (c) a mefloquine prodrug or (d) a mefloquine metabolite.

16. A method according to claim 15 in which the therapeutically effective amount is an amount that will produce a blood concentration of mefloquine of 10 μM or less.

17. A composition for treating cancer comprising (i) an effective amount of a compound having the formula (I): 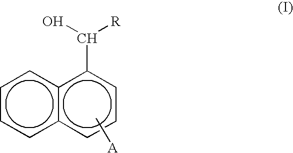
in which A represents from one to three groups selected from halogen and trifluoromethyl, and in which the quinoline ring is optionally further substituted by one or more other moieties, and R is (a) NR₁R₂ in which R₁ and R₂ are independently hydrogen or C₁-C₄ alkyl; (b) 2-piperidyl, (c) 2-pyridyl, and (d) 5-(ethyl or vinyl)-quinuclidin-4-yl; an enantiomer of such a compound; a pharmaceutically acceptable salt of such a compound or of an enantiomer thereof; a prodrug of such a compound or of an enantiomer thereof; a metabolite of such a compound or of an enantiomer thereof; or a mixture of two or more of the foregoing, and (ii) a pharmaceutically acceptable carrier.

18. A composition according to claim 17 in which A represents from one to three chloro or trifluoromethyl groups, and R is (a) NR₁R₂ in which R₁ and R₂ are independently hydrogen or C₃-C₄ alkyl; (b) 2-piperidyl, (c) 2-pyridyl, and (d) 5-(ethyl or vinyl)-quinuclidin-4-yl.

19. A composition according to claim 17 in which the quinoline ring is further substituted by a methoxy, methyl, phenyl, halophenyl or trifluoromethyl group.

20. A composition according to claim 17 in which the quinoline ring is substituted by from one to three groups selected from halogen and trifluoromethyl and is not further substituted.

21. A composition according to claim 17 in which the compound is selected from mefloquine, enantiomers of mefloquine; prodrugs of mefloquine or of its enantiomers; metabolites of mefloquine or of its enantiomers; pharmaceutically acceptable salts of mefloquine, of mefloquine enantiomers, of mefloquine prodrugs or of mefloquine metabolites, and mixtures thereof.

22. A composition according to claim 17 that is in a form suitable for oral administration.

23. A kit for treating cancer comprising a composition according to claim 17.

24. A kit according to claim 23 in which A represents from one to three chloro or trifluoromethyl groups, and R is (a) NR₁R₂ in which R₁ and R₂ are independently hydrogen or C₃-C₄ alkyl; (b) 2-piperidyl, (c) 2-pyridyl, and (d) 5-(ethyl or vinyl)-quinuclidin-4-yl.

25. A kit according to claim 23 in which the quinoline ring is further substituted by a methoxy, methyl, phenyl, halophenyl or trifluoromethyl group.

26. A kit according to claim 23 in which the quinoline ring is substituted by from one to three groups selected from halogen and trifluoromethyl and is not further substituted.

27. A kit according to claim 23 in which the compound is selected from mefloquine, enantiomers of mefloquine; prodrugs of mefloquine or of its enantiomers; metabolites of mefloquine or of its enantiomers; pharmaceutically acceptable salts of mefloquine, of mefloquine enantiomers, of mefloquine prodrugs or of mefloquine metabolites, and mixtures thereof.
